(19) United States
(12) Patent Application Publication
(10) Pub. No.: US 2021/0374891 A1
(43) Pub. Date: Dec. 2, 2021

(54) NETWORK TRACKING AND ENFORCEMENT OF SOCIAL DISTANCING PROTOCOLS

(71) Applicant: DISH Wireless L.L.C., Englewood, CO (US)

(72) Inventors: Anand Menon, Lone Tree, CO (US); Max Stephen Gratton, Parker, CO (US); Matthew Polson, Parker, CO (US)

(21) Appl. No.: 17/020,246

(22) Filed: Sep. 14, 2020

Related U.S. Application Data
(60) Provisional application No. 63/029,885, filed on May 26, 2020.

(51) Int. Cl. G06Q 50/26; H04W 4/029; H04W 4/02; G08B 21/02; G06Q 30/00; G16H 50/80; G06F 16/23

(52) U.S. Cl. CPC G06Q 50/265; H04W 4/029; H04W 4/023; G06N 20/00; G06Q 30/0185; G16H 50/80; G06F 16/2379; G08B 21/02

(57) ABSTRACT

Novel techniques are described for protocol tracking and/or enforcement (PT&E) of social distancing protocols in communication networks. For example, PT&E data can be received and aggregated from a large number of user mobile devices via wireless communication networks. The PT&E data can be analyzed against a pre-defined social distancing protocol to detect candidate non-compliance events. The social distancing protocol is stored in association with predefined exclusions to application of the social distancing protocol. The candidate non-compliance events can be analyzed against the predefined exclusions to determine whether the detected events are excluded from consideration as a non-compliant condition. The detected candidate non-compliance events and/or non-compliant conditions can then support various features, such as warning individuals and/or organizations of compliance concerns, contact tracing responsive to non-compliant conditions, compliance scoring for entities, social grading for individual compliance, heat mapping of compliance-related data, etc.

### CROSS-REFERENCE TO RELATED APPLICATIONS

This application claims priority to U.S. Provisional Patent Application No. 63/029,885, filed on May 26, 2020, entitled “Network Tracking And Enforcement Of Social Distancing Protocols,” the disclosure of which is hereby incorporated by reference in its entirety for all purposes.

### FIELD

This invention relates generally to communication networks, and, more particularly, to tracking and enforcement of social distancing protocols in communication networks.

### BACKGROUND

In a number of contexts, there is a desire to promulgate, enforce, and/or track certain social distancing rules. One such context is when a population is trying to limit the spread of a certain pathogen. At different times and in different places around the world, humans are exposed to many different communicable pathogens. Such viruses, bacteria, and other pathogens can be transmitted across populations, thereby spreading illnesses, even when there is no direct physical contact between individuals. The spread of these pathogens can often be through contact with bodily fluids transmitted by an infected individual when the individual coughs, sneezes, speaks, touches surfaces, etc. For example, the past decade has seen global pandemics caused by the rapid spread of respiratory viruses in the coronavirus family, including the so-called novel coronavirus (2019-nCoV, or COVID-19), the so-called Middle East respiratory syndrome (MERS), and the so-called Severe acute respiratory syndrome (SARS). With such pathogens, even asymptomatic individuals are often still contagious. These and other factors can frustrate individual attempts to avoid infecting, or becoming infected by, others.

It often takes months or years before vaccines can be developed, tested, and deployed to counter the spread of novel pathogens, or pathogen strains. Meanwhile, in addition to having deleterious impacts on the health of those infected with the pathogens, such rapid propagation can have many undesirable secondary effects, such as overwhelming of medical infrastructures, mass hysteria, and economic downturn. It is thus desirable to individuals, businesses, regulators, governments, and other entities to find sufficiently simple and reliable techniques to limit or slow the spread of pathogens. In the wake of the COVID-19 pandemic, governments and businesses around the globe adopted social distancing and related protocols, such as requiring (or at least recommending) that individuals maintain a distance of at least 6 feet (or 2 meters) from others, and that personal protective equipment (PPE) be worn in public areas. Despite the seeming simplicity of such protocols, it can be difficult for individuals and entities to know whether they are in compliance, to communicate and receive compliance information about others, and/or to remain updated with relevant information to aid in compliance.

### SUMMARY

Among other things, embodiments provide novel systems and methods for protocol tracking and/or enforcement (PT&E) of social distancing protocols in communication networks. For example, a back-end system can be configured to receive PT&E data from a large number of user mobile devices via wireless communication networks, either directly or through one or more aggregator devices. The PT&E data is associated with at least one pre-defined social distancing protocol stored in association with a set of exclusions to application of the social distancing protocol. For example, the social distancing protocol can be for mitigating spread of a contagious pathogen, mitigating spread of protected information, etc. The PT&E data can be aggregated and analyzed by the back-end system to detect one or more candidate non-compliance events, such as any interactions between user mobile devices, or other events that conflict with one or more guidelines defined by the social distancing protocol. The back-end system can further detect whether any of the candidate non-compliance events are excluded from consideration as a non-compliant condition, based on applying the defined set of exclusions. The detected candidate non-compliance events and/or non-compliant conditions can then be used to provide various features, such as warning individuals of compliance concerns, contact tracing responsive to non-compliant conditions, compliance scoring for entities, social grading for individual compliance, etc.

This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings, and each claim.

The foregoing, together with other features and embodiments, will become more apparent upon referring to the following specification, claims, and accompanying drawings.

### BRIEF DESCRIPTION OF THE DRAWINGS

The present disclosure is described in conjunction with the appended figures:

FIG. 1 shows a network environment as a context for various embodiments;

FIG. 2 shows a block diagram of an illustrative user mobile device, according to various embodiments described herein;

FIG. 3 shows a block diagram of an illustrative local aggregator device and an illustrative back-end system, according to various embodiments described herein;

FIG. 4 shows an illustrative public park environment in which various individuals are recreating in different ways;

FIG. 5 shows an illustrative office environment in which individuals are engaged in a typical work environment;

FIG. 6 provides a schematic illustration of one embodiment of a computer system that can implement various system components and/or perform various steps of methods provided by various embodiments;

FIG. 7 shows a flow diagram of an illustrative method for tracking of social distancing protocols to mitigate proximity-based communicability, according to various embodiments; and

FIG. 8 shows an illustrative simplified computational system for a computing protocol compliance events and related data, according to various embodiments.

In the appended figures, similar components and/or features may have the same reference label. Further, various components of the same type may be distinguished by following the reference label by a second label (e.g., a lower-case letter) that distinguishes among the similar components. If only the first reference label is used in the specification, the description is applicable to any one of the similar components having the same first reference label irrespective of the second reference label.

### DETAILED DESCRIPTION

Embodiments of the disclosed technology will become clearer when reviewed in connection with the description of the figures herein below. In the following description, numerous specific details are set forth to provide a thorough understanding of the present invention. However, one having ordinary skill in the art should recognize that the invention may be practiced without these specific details. In some instances, circuits, structures, and techniques have not been shown in detail to avoid obscuring the present invention.

In the wake of the COVID-19 pandemic, governments and businesses around the globe adopted social distancing and related protocols, such as requiring (or at least recommending) that individuals maintain a distance of at least 6 feet (or 2 meters) from others, that certain types of gatherings be limited to certain numbers of individuals, and that personal protective equipment (PPE) be worn in public areas. Despite the seeming simplicity of such protocols, it can be difficult for individuals and entities to know whether they or others are in compliance, to communicate and receive compliance information about others, and/or to remain updated with relevant information to aid in compliance.

Some reasons that individual compliance can be difficult relate to individuals conventionally having no practical way to reliably estimate, anticipate, or extricate themselves from contexts in which they are less than a protocol-defined distance away from others. For example, individuals standing in a common area, sitting in a board room, using a public restroom, or the like, may have a difficult time knowing whether they are properly distanced from others, particularly when different distances are permitted in different contexts (e.g., based on time of exposure, whether the exposure is indoors or outdoors, whether the exposure is to permitted individuals, etc.). As another example, as individuals move, each individual may not be able to anticipate, or may not otherwise notice, the manner in which their locations change with respect to the locations of others. As another example, protocols may be difficult for individuals to track as rules are added or removed, and/or as different rules are applied to different contexts. Entity-level compliance (e.g., whether at a business level, government level, etc.) can also be difficult, as it may be conventionally impractical for entities to reliably monitor and aggregate compliance of their individual stakeholders (e.g., employees, customers, etc.), and/or to communicate compliance-related information in a meaningful way.

Some conventional approaches exist for providing location tracking and related features. For example, conventional global positioning satellite (GPS) applications can enable smart devices to derive their locations, typically within a few meters. Various conventional social networking applications can enable individuals to see when friends are in a same general vicinity, or the like. One company recently began to require its employees to wear a smart watch that uses Bluetooth technology to detect and indicate (e.g., buzz) when it is within six feet of another such watch. Still, each of these conventional technologies has limitations that limit their efficacy for reliable tracking and enforcement of social distancing protocols in communication networks, particularly as compared with novel approaches described herein. For example, such conventional approaches tend to be locked into a particular protocol (e.g., they are set to inform the user of entering a predetermined, fixed radius of another individual), and they tend only to address limited aspects of compliance (e.g., they focus only on detecting proximity).

Among other things, embodiments provide novel systems and methods for tracking and/or enforcement of social distancing protocols in communication networks. Though certain embodiments are described with reference to a subset of features, it will be appreciated that implementations can combine features from multiple (even all) described embodiments. Further, the term “social distancing” is intended to include other related protocols. For example, compliance with a social distancing protocol may, in a particular context, involve inaction (e.g., remaining in a location to avoid non-compliance), or taking action not related to physical location (e.g., wearing a respiratory mask and/or other PPE).

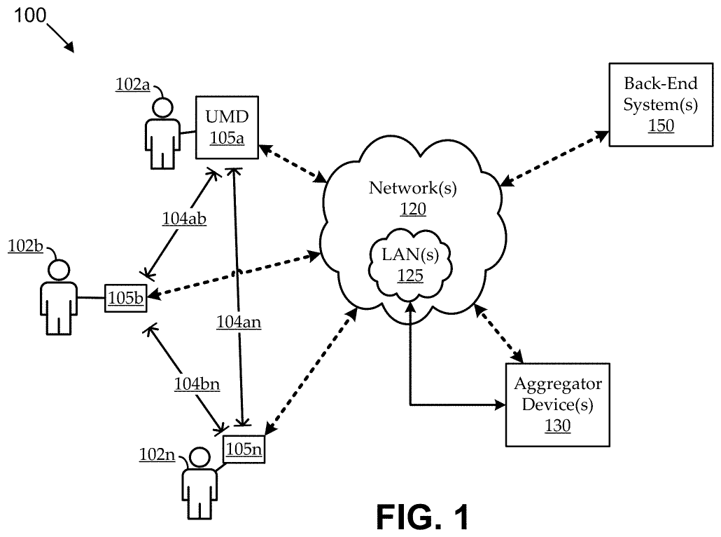

Turning to FIG. 1, a network environment 100 is shown as a context for various embodiments. The network environment 100 includes a back-end system 150 in communication with a number of user mobile devices 105 over one or more networks 120. In some embodiments, user mobile devices 105 are directly in communication with the back-end system 150 via the network(s) 120, such as over one or more local or remote networks, the Internet, etc. In other embodiments, some or all user mobile devices 105 are in communication with one or more local aggregator devices 130 over one or more local area networks (LANs) 125, and the one or more local aggregator devices 130 are in communication with the back-end system 150 over the same or other networks 120. In some embodiments, the local aggregator devices 130 are part of the back-end system 150.

As described herein, the user mobile devices 105 can effectively be proxies for users 102. For example, a user's 102 location information, travel patterns, etc. can be obtained by analyzing corresponding information from one or more user mobile devices 105 known to be associated with that user 102. This information can then be analyzed with respect to corresponding information for other users 102 to determine and/or provide relevant information described herein, such as relating to the user's location, proximity to other user's, social distancing-related behaviors, etc. Such information can be utilized for monitoring and/or informing behavior of individuals and organization, monitoring and/or informing compliance with behavioral protocols, and/or the like.

Many communicable pathogens can spread quickly through populations based on a variety of factors, including types of interpersonal contact. As used herein, terms like “pathogen,” “contagion,” “communicable pathogen,” etc. are intended broadly to include any virus, bacterial, or other pathogen that can be transmitted from one individual to another through contact or proximity, potentially resulting in a health condition. Some examples include seasonal flu and coronaviruses. In some cases, these pathogens become serious health risks, at least to certain portions of the population. Over a short time window, a single individual can be in close contact with large numbers of diverse individuals over a large geographical area. For example, a typical day of business travel can involve an individual taking a crowded train to the airport, walking through the crowded airport, taking a crowded flight to another city, having meetings and meals in multiple locations in the other city, and staying in a crowded hotel that evening. In that single day, the individual may have been in relatively close contact with hundreds of people in multiple distant locations, potentially becoming infected with, and potentially transmitting, many different pathogens. Further, as interpersonal contact patterns can grow exponentially (e.g., one individual contacts multiple individuals, who each contact multiple individuals, and so on), highly contagious pathogens can quickly become global pandemics.

Some ways to slow the spread of some such pathogens include informing about and/or enforcing certain behaviors, such as increasing certain hygienic practices (e.g., washing of hands, boiling of water, etc.), avoiding certain types of contact (e.g., quarantining infected individuals, advising self-quarantining of at-risk populations, limiting large gatherings, etc.), and encouraging proactive medical interventions (e.g., vaccination, testing, etc.). Conventionally, such behavior-based approaches tend to be limited in a number of ways, due at least in part to a lack of reliable, real-time, and relevant information. For example, many people can become infected with a highly contagious pathogen and may experience no symptoms or mild symptoms, while still being able to infect others. Even when an infected individual exhibits significant symptoms, by the time such an individual is diagnosed with a particular contagious virus, the individual may already have been carrying and passing along the virus for days. At that point, quarantining the individual can only help limit further spread of the virus. Conventionally, it tends to be impractical to identify and/or inform populations of individuals who may have contracted the pathogens from that infected individual; meanwhile, those potentially infected populations continue to contact and potentially infect additional populations.

For at least these reasons, governments, regulators, and other organizations have tended to rely more heavily on precautionary and prophylactic measures, such as encouraging and/or enforcing social distancing behaviors. As noted above, “social distancing” behaviors can refer to maintaining of a predefined (e.g., protocol-defined) minimum physical distance from others. For example, in the illustrated environment 100, a number of users 102 each have at least one associated user mobile device 105, and each of those user mobile devices 105 is some distance 104 from other user mobile devices 105. Such behaviors, as described herein, can also be extended in certain contexts to broadly include other related behaviors, such as use of personal protective equipment (PPE) when around others, regular monitoring of certain symptomatic indicators (e.g., body temperature), regular performance of hygienic behaviors (e.g., hand washing, use of disinfecting wipes on surfaces, etc.), reporting or checking-in at certain intervals, etc. In some instances, reactionary measures can be added to such precautionary and prophylactic measures. In response to detection of an infected individual or group of individuals in a particular location, heightened protocols can be imposed on related groups and/or locations. In one example, if one or more cases of a pathogen are detected in individuals in a particular location (e.g., a school, county, etc.), particular quarantine rules may be imposed for any individuals known to have been in that location over a certain time period, for any individuals known to have been contact with the infected individuals, etc. In another example, if multiple new cases of a pathogen are detected close in time in a particular location, the entire location may be placed under lock-down; individuals may be restricted from entering and/or leaving a defined area.

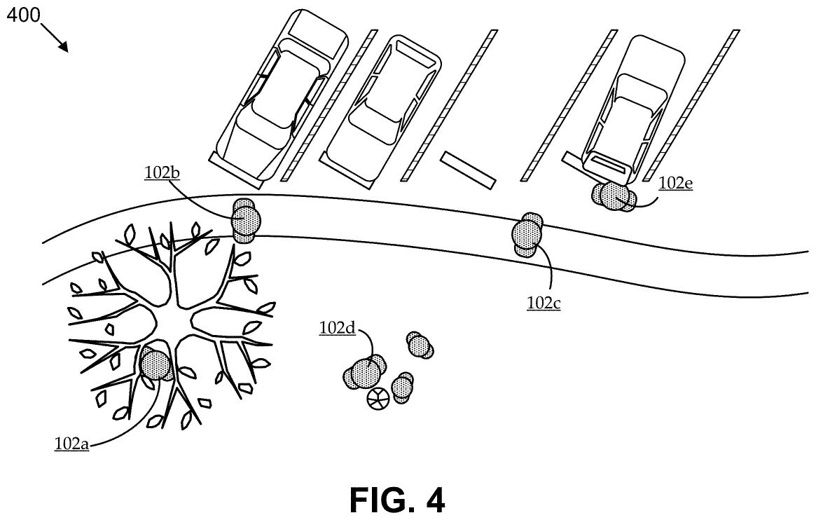

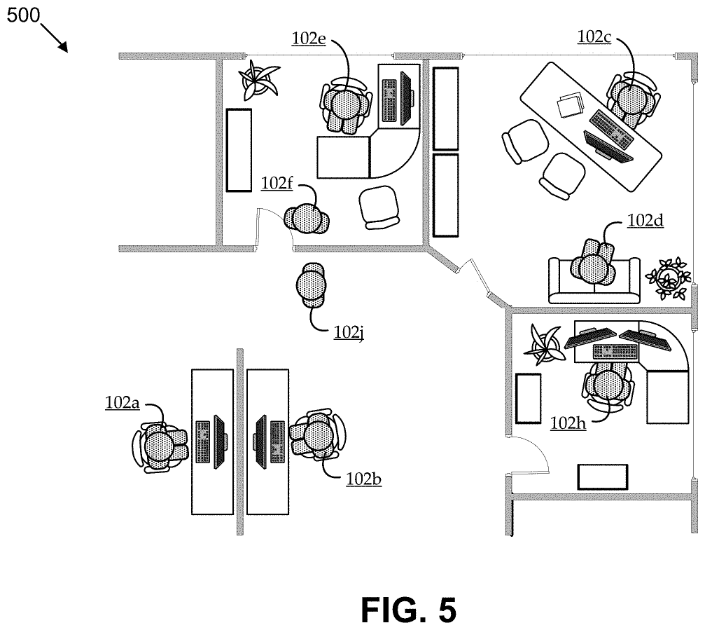

In many contexts, it is simple for individuals to know whether they are complying with social distancing behaviors. However, many factors can make such compliance more difficult or uncertain. For the sake of illustration, FIGS. 4 and 5 shows two typical environments to demonstrate some such factors. FIG. 4 shows an illustrative public park environment 400 in which various individuals are recreating in different ways. As shown, one individual 102a is sitting under a tree reading, two individuals 102b and 102c are walking toward each other on a relatively narrow path, a parent (individual 102d) and two children are playing ball in the grass, cars are parked in an adjacent lot, and one individual 102e is retrieving something from the trunk of his car. Even in context of a simple social distancing protocol that prescribes maintaining a minimum distance of six feet from others, such an environment creates practical complexities for compliance. The family playing in the grass is clearly not maintaining the prescribed social distancing, but they are members of a same household. If any of them is infected, it is highly likely the others are also already infected; so there may be no practical reason to enforce social distancing for such a case. The two individuals on the path are approaching one another, and one will have to move off of the path to maintain prescribed social distancing. However, there may be no way to do that without one of the individuals either moving toward the family (and potentially creating another non-compliant situation), or moving toward the individual in the parking lot. Further, the individuals will likely only be in brief passing contact, which may be of appreciably reduced concern for certain pathogens. The individual sitting behind the tree may be trying to stay distant from everyone. In fact, however, the individual may end up less than the prescribed minimum distance from, and also relatively hidden from, the individuals passing on the path and/or the family. Again, for a pathogen that tends to be communicated by droplets in the air, it may be highly unlikely that an individual sitting and reading under a tree will infect others, even nearby; but it is may be more likely for such an individual to become infected by those walking, running, or playing nearby. The individual in the parking lot may have just arrived, and may not be paying any attention yet to nearby individuals, such as those walking on the path. In any event, with the individual facing away from others, the likelihood of pathogen communication to or from that individual may be very low. Further, though not shown, there may be individuals sitting in cars parked in the adjacent lot, and those individuals may be well within the prescribed minimum distance from the individuals passing on the path and/or from other individuals in other cars. However, the proximity of individuals in cars to others outside the cars may only be of concern as the individuals enter or exit their cars, when the individuals leave their car windows or doors open, etc. (e.g., an airborne pathogen cannot be communicated through a solid physical barrier, such as a window or a door).

To further illustrate these complexities, FIG. 5 shows an illustrative office environment 500 in which individuals are engaged in a typical work environment. The illustrated environment includes multiple offices and cubicles. Two individuals (102a and 102b) are illustrated as sitting in adjacent cubicles facing each other and separated by a distance of less than a prescribed minimum distance, but they are separated by a five-foot-tall cubicle barrier. While the individuals are seated, they may be very likely protected from each other with respect to communication of certain types of pathogens. However, some types of pathogens may be more likely to spread even in such an environment, and/or the individuals may be less protected while standing. A separate concern can arise if those cubicle workspaces are shared. Even if individuals using those workspaces are never within the minimum prescribed distance from each other, some pathogens may remain on surfaces of the shared workspace from one user to the next, potentially leading to the spread of a pathogen.

In the corner office, individuals (102c and 102d) are sitting sufficiently far away from each other to comply with a social distancing protocol, but extended contact may increase the risk. In comparison, in the adjacent office to the left, the individuals (102e and 102f) may be unable to stand far enough apart (e.g., the office is too small), but the distance is close to the prescribed minimum and contact may be extremely brief. Further, individuals are illustrated as certainly less than the prescribed minimum distance from others, but also without any concern of pathogen spread. For example, individual 102d on a couch in the corner office is relatively close to individual 102h working in the adjacent office below, but they are separated by a wall. Similarly, an individual 102j in the common space outside the offices is relatively close to an individual 102f standing in the adjacent office to the left, but they are also separated by a wall. Notably, however, as individual 102j continues to walk towards the left, or if individual 102f exits the office, a situation quickly can arise in which individuals 102f and 102j are similarly proximate, but are no longer separated by a wall.

The above examples are intended only to illustrate some of the many scenarios in which social distancing protocols may be desired, but in which practical complexities may arise relating to the adoption, compliance, and enforcement of such protocols. Additional related complexities arise when applying such protocols on larger scales, such as at a business level, a neighborhood level, a state or federal level, etc. For example, it can conventionally be impractical or impossible to reliably incentivize, monitor, and/or enforce compliance at those levels. Embodiments described herein include novel techniques for using the user mobile devices 105 and one or more back-end system(s) 150 (e.g., in addition to, or integrated with, one or more local aggregator devices 130) to manage, inform, enforce, monitor, track, score, and/or otherwise interface with social distancing protocols.

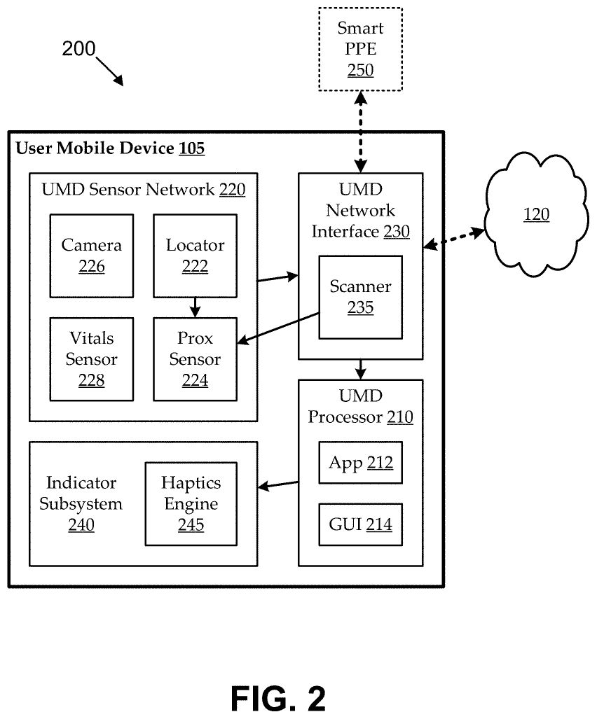

FIG. 2 shows a block diagram of an illustrative user mobile device 105, according to various embodiments described herein. The user mobile device 105 can be an implementation of the user mobile device 105 shown in FIG. 1. Each user mobile devices 105 can be, or can include, any suitable networked devices that are associable with a particular user 102 and include location tracking capability. In some implementations, some or all user mobile devices 105 are smart phones. In other implementations, some or all user mobile devices 105 are wearable devices. For example, the user mobile devices 105 can be implemented as smart watches, smart wristbands, fitness trackers, medical trackers, etc. In some embodiments, the user mobile device 105 is implemented as smart PPE 250, such as a respiratory mask may have components of the user mobile device 105 integrated therein. Alternatively the smart PPE 250 can have fewer components (e.g., dedicated sensor and network interface components), and can be configured to interface (e.g., wirelessly) with user mobile devices 105.

As illustrated, each user mobile device 105 can include some or all of a user mobile device (UMD) processor 210, a UMD sensor network 220, a UMD network interface 230, and an indicator subsystem 240. The UMD processor 210 can support a graphical user interface (GUI) 214 (e.g., provided via input/output components of the user mobile device 105 (not shown), such as a touchscreen, display, keypad, etc.). The UMD processor 210 can also support one or more dedicated applications 212 to implement certain features described herein, such as alert features, tracking features, reporting features, etc. The UMD processor 210 can be implemented using one or more central processing units CPUs, application-specific integrated circuits (ASICs), application-specific instruction-set processors (ASIPs), graphics processing units (GPUs), digital signal processors (DSPs), field-programmable gate arrays (FPGAs), programmable logic devices (PLDs), controllers, state machines, microcontroller units, reduced instruction set (RISC) processors, complex instruction set (CISC) processors, microprocessors, or the like, or any combination thereof.

In some embodiments, the UMD processor 210 can direct operation of the indicator subsystem 240. The indicator subsystem 240 can include any suitable indicator components, such as audio components (e.g., buzzers, speakers, etc.), illuminating components (e.g., light-emitting diodes), display screens, etc. In some implementations, the indicator subsystem 240 includes a haptics engine 245. For example, as described herein, when the user mobile device 105 detects that it is too close to (i.e. less than a protocol-prescribed minimum distance away from) another user mobile device 105, the indicator subsystem 240 can cause the user mobile device 105 to vibrate or pulse, cause a visual indicator to illuminate or change color, cause an audible indicator to play a sound or buzz, etc. In some implementations, the indicator subsystem 240 provides additional information, such as by guiding the user into compliance. For example, the haptics engine 245 and/or other components of the indicator subsystem 240 can be used to point the user in an optimal direction for distancing the user from others. In some implementations, the haptics engine 245 and/or other components of the indicator subsystem 240 are configured to aid visually impaired and/or hearing impaired individuals. For example, the haptics engine 245 can provide tactile information to guide visually impaired individuals away from non-compliant locations, or audio components can provide audible information to guide hearing impaired individuals away from non-compliant locations.

In some implementations, the indicator subsystem 240 and/or the GUI 214 are used to communicate compliance data, reminders (e.g., about checking in, about proper hygienic practices, etc.), and/or other information. As regulations associated with social distancing protocols change over time, location, etc., those changes can be pushed to the user mobile devices 105, and updates can be displayed via the indicator subsystem 240 and/or the GUI 214. As one example, wearing of masks and/or other PPE may only be required in certain locations at certain times, and the user mobile devices 105 can indicate (via the indicator subsystem 240 and/or the GUI 214) when a user has entered an area with a particular PPE requirement.

Embodiments of the UMD sensor network 220 include any suitable sensor components. For example, embodiments of the UMD sensor network 220 can include one or more locator sensors 222, proximity sensors 224, cameras 226, and/or vitals sensors 228. Though illustrated as integrated within the user mobile device 105, embodiments of the UMD sensor network 220 can include any suitable on-board sensor components, and/or can include any suitable on-board components for interfacing with off-board sensor components. For example, the user mobile devices 105 may be primarily integrated into a smart phone, and the vitals sensor 228 may be integrated within a fitness tracker bracelet. As such, components of the UMD sensor network 220 on-board the smart phone may include interface components to communicate (e.g., wirelessly) with the separate vitals sensor 228. Embodiments of the locator sensors 222 can include any suitable sensors and related components for determining location information for the user mobile device 105, such as one or more accelerometers, gyroscopic components, global positioning satellite (GPS) receivers, etc. The locator sensors 222 can determine any or all of present location, heading (i.e., pointing direction), direction of movement, speed, altitude, etc.

Embodiments of the proximity sensors 224 can determine proximity of the user mobile device 105 to one or more other user mobile devices 105 in any suitable way. In some implementations, the proximity sensors 224 can detect a wireless signal from another user mobile device 105 or wireless fixed beacon and measure a signal strength. An estimate of proximity can be calculated from the signal strength. In other implementations, the proximity sensors 224 can communicate signals structured to provide proximity information. For example, periodic signals can be used to detect Doppler shifts, beacon signals can be used to detect round trip timing, etc. In some embodiments, the UMD network interface 230 includes a scanner 235, such as a radiofrequency scanner, to scan through multiple radio and/or other technologies to look for proximate signals of different types. For example, software-defined networking (SDN), multiple dedicated chipsets, or other techniques can be used to scan multiple radio technologies and/or frequency bands. Such scanning can enable the user mobile device 105 to detect a greater variety of potentially proximate devices affiliated with users.

Embodiments of the cameras 226 and the vitals sensors 228 can be used to obtain any relevant information related to an individual's context to support various features described herein. In some implementations, a social distancing protocol recommends or requires individuals to use the camera 226 to capture images to support or demonstrate compliance. For example, the camera 226 can be used to show the individual properly using PPE (e.g., wearing a mask in a public area), checking into a diagnostic facility, setting up a workstation with appropriate protective components (e.g., a plexiglass divider, where appropriate), etc. In other implementations, the vitals sensors 228 periodically or continually monitor and/or report information about an individual user, such as the individual's body temperature, heart rate, pulse, pulse-oxygen level, etc. In some embodiments, the vitals sensors 228 (or other suitable components) can be used to detect whether an individual is using the user mobile device 105. For example, a liveness sensor (e.g., sensing bio-capacitance, blood flow, body temperature, etc.) can be used to detect that the individual is wearing or carrying the user mobile device 105. In other implementations, the same and/or additional sensors can be used to capture and/or report other information about an individual, such as the individual's general activity level (e.g., fitness level), diet, etc. In other implementations, the same and/or additional sensors can be used to capture and/or report other information about environmental conditions, such as ambient temperature, ambient humidity, levels of pollutants or toxins, etc.

In at least one implementation, data collected by the proximity sensor 224 can be used to monitor for things like proper hand-washing. In this example, proximity sensor 224 may be detect a user entering a restroom and coming in close proximity to a beacon located near a toilet. A second wireless beacon may be located near a sink, and the UMD processor 210 may determine that the user did not stop near the sink beacon, indicating that the user likely did not wash their hands. Likewise, the proximity sensor 224 may measure a signal from the sink beacon, but the processor 210 may measure that the user did not spend enough time near the sink, such that a proper amount of time was not spent handwashing. The indicator subsystem 240 may provide appropriate warning to the user as a reminder to return and wash their hands. Likewise, such data may be transmitted to the cloud and collected for compliance purposes.

In at least one embodiment, a sensor (e.g., a proximity sensor) is located in or near a sink, soap dispenser, or any other suitable location of a restroom, kitchen area, etc. Such a sensor can measure how long the user spends near the sink and can provide an audible or visual warning if the a proper amount of time was not spent handwashing. The sensor can be configured to start an audible or visual timer that specifies how long as user has been washing their hands, or how long the need to continue washing their hands for effective washing. For example, a proximity sensor data in a soap dispenser may start the washing timer, and sensor data from a sink or faucet may measure whether the user is still present near the sink, or whether the water has been shut off (either with manual or motion activated water faucets). Such data can be processed by a processing system to determine whether to present an audible or visual warning to a user. For example, an audible violation warning can be sounded if a user does not spend sufficient time hand washing; and/or an audible success tone can be sounded if a user does spend sufficient time hand washing. Some implementations can include additional sensors to gather and/or confirm related information. For example, a water flow sensor can detect whether and for how long water is flowing from a sink, a microphone can detect the sound of flowing water, a pressure pad on the floor can detect whether a user is standing next to the sink, etc.

Embodiments of the UMD network interface 230 can facilitate communicative coupling with the one or more networks 120. As shown in FIG. 1, such communicative coupling can enable communications between a user mobile device 105 and other user mobile devices 105, between the user mobile device 105 and one or more local aggregator devices 130, between the user mobile device 105 and one or more back-end systems 150, etc. Also as shown in FIG. 1, embodiments of the one or more networks can include one or more LANs 125. The UMD network interface 230 can include any suitable network components to communicate with any suitable types of networks 120. For example, the UMD network interface 230 of each user mobile devices 105 can include a wireless fidelity (WiFi) transceiver radio or interface, a Bluetooth transceiver radio or interface, a Zigbee transceiver radio or interface, an Ultra-Wideband (UWB) transceiver radio or interface, a WiFi-Direct transceiver radio or interface, a Bluetooth Low Energy (BLE) transceiver radio or interface, and/or any other wireless network transceiver radio or interface that allows the user mobile devices 105 to communicate with the network(s) 120. The user mobile devices 105 can be identifiable in the network(s) 120 using any suitable technology, including, for example, a media access control (MAC) address, an Internet protocol (IP) address, etc. In some implementations, a user mobile device 105 can communicate with the network(s) 120 via one or more other device. For example, a user mobile devices 105 is in short-range wireless communication with a second user mobile device 105 and/or an local aggregator device 130, which is in communication with the network(s) 120. In some cases, a user may attempt to shut off the short-range wireless communication network interface. In that case, the user mobile device 105 may be designed to emit a warning sound indicator to warn the user or other people around that the proximity sensor 224 is not operational.

Embodiments of the network(s) 120 can include any type of wired or wireless network links, or combinations thereof. For example, the network(s) 120 can include one or more of a cable network, a wireline network, an optical fiber network, a telecommunications network, an intranet, an Internet, a local area network (LAN), a wide area network (WAN), a wireless local area network (WLAN), a metropolitan area network (MAN), a wide area network (WAN), a public telephone switched network (PSTN), a Bluetooth network, a ZigBee network, a near field communication (NFC) network, or the like, or any combination thereof. In some embodiments, the network(s) 120 include one or more network access points, such as wired or wireless network access points (e.g., base stations and/or internet exchange points).

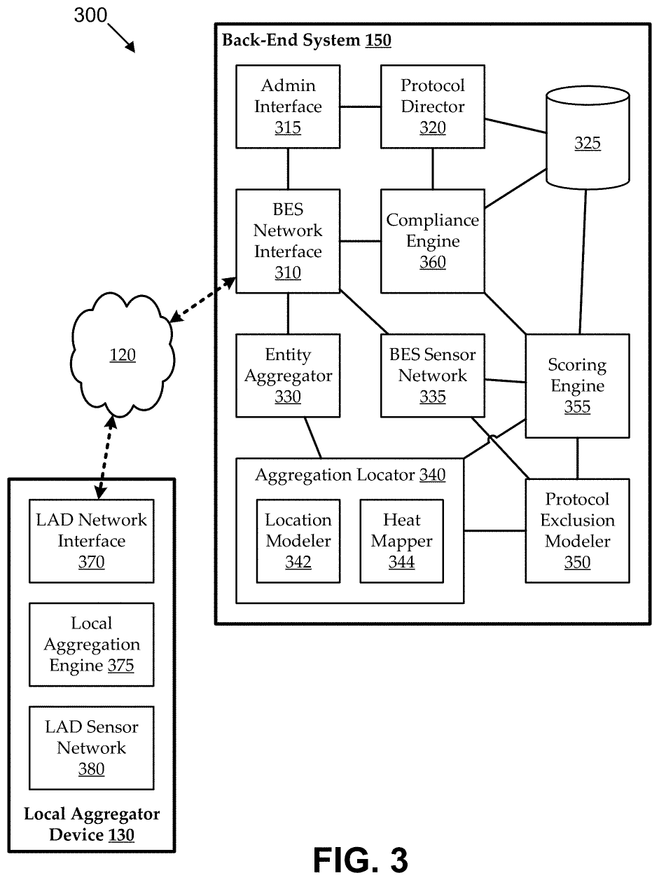

FIG. 3 shows a block diagram of an illustrative local aggregator device (LAD) 130 and an illustrative back-end system (BES) 150, according to various embodiments described herein. The BES 150 and the LAD 130 can be implementations of the back-end system 150 and the local aggregator device 130 of FIG. 1, respectively. As described above, some embodiments do not include a LAD 130, and other embodiments integrate the LAD 130 and the BES 150 in a single computational environment (e.g., in one or more physical or logical devices). Embodiments of the LAD 130 can include any suitable components for aggregating data from multiple user mobile devices 105 in a local area, and/or for acting as a central communication node to enable a LAN for multiple user mobile devices 105 in a local area. In general, either or both of the LAD 130 and the BES 150 can be implemented in a single computational environment, or distributed over multiple computational environments; and either or both of the LAD 130 and the BES 150 can be implemented in a single device (e.g., within a single housing), or distributed over multiple devices (e.g., coupled directly or indirectly in any suitable manner). For example, any features described herein with reference to a particular component can be performed by another suitable component in an alternative implementation; and any features described with reference to either the LAD 130 or the BES 150 can additionally or alternatively be performed by the other.

Descriptions of the LAD 130 and/or the BES 150 obtaining data from the user mobile devices 105 and/or various types of sensor networks can generally be referring to protocol tracking and enforcement (PT&E) data. PT&E data can generally include any type of data that is useful in making determinations relating to social distancing protocol tracking and/or enforcement. The PT&E data is generally related to output from one or more sensors. In some implementations, PT&E data can include position-related information obtained from sensors of the user mobile devices 105, such as a current GPS position data, accelerometer and/or gyroscopic data, speed and/or direction of travel data, heading data, altitude data, signal measurement data (e.g., for triangulation of position), etc. In some implementations, PT&E data can include information environmental data from sensors integrated with the user mobile devices 105 and/or separate from the user mobile devices 105, such as temperature data, humidity data, wind speed data, still and/or video imagery of an area, etc. In some implementations, PT&E data can include information from sensors integrated with smart PPE, such as data indicating whether the PPE is being used. In some implementations, PT&E data can include health information, or the like, obtained from sensors integrated in the user mobile devices 105, in smart PPE, in wearable sensor devices, etc., such as heart rate data, body temperature data, etc.

Embodiments of the LAD 130 can include a LAD network interface 370, a local aggregation engine 375, and/or a LAD sensor network 380. Embodiments of the LAD network interface 370 can include any suitable features and components to enable communicative coupling with user mobile devices 105 and/or BESs 150 via the one or more networks 120. In some implementations, the LAD 130 is configured to provide its own one or more LANs 125 for communicating at least with user mobile devices 105. Some embodiments of the LAD 130 are implemented in a set-top box (e.g., a television receiver appliance), a digital video recorder appliance, a video game console appliance, LAN router appliance, etc. In some embodiments, the LAD network interface 370 is configured to provide a shared network resource for the user mobile devices 105 that extends their networking capability. For example, each user mobile device 105 can include a relatively low-power, low-bandwidth, and/or high-latency wireless link to the LAD 130, and the LAD 130 can support a relatively high-power, high-bandwidth, and/or low-latency wireless link to the BES 150.

Embodiments of the local aggregation engine 375 include any suitable hardware and/or software to aggregate data from multiple user mobile devices 105 in communication with the LAD 130 at a single time, or over a time window. In some embodiments, the local aggregation engine 375 aggregates the data, and sends aggregated data to the BES 150. In other embodiments, the local aggregation engine 375 obtains data in a manner that facilitates aggregation by the BES 150 (i.e., the LAD 130 sends non-aggregated data to the BES 150, and the BES 150 aggregates the data).

Embodiments of the LAD sensor network 380 can include any sensors to provide or support additional features. In some implementations, some or all of the sensors are included on-board the LAD 130 as part of the LAD sensor network 380. In other implementations, some or all of the sensors are off-board the LAD 130, and they are considered part of the LAD sensor network 380 by virtue of the LAD sensor network 380 being in communication with those sensors and being capable of receiving (e.g., and processing, aggregating, etc.) data from those sensors. In some embodiments, the LAD sensor network 380 includes one or more environmental sensors to track time of day, ambient conditions (e.g., temperature, wind, humidity, light intensity, etc.), pollutant levels, etc. In other embodiments, the LAD sensor network 380 includes one or more cameras, such as by being in communication with a security camera, or other closed caption television (CCTV), network and can obtain video information to support certain features. For example, collected CCTV footage can be used to monitor, report, or establish compliance with social distancing protocols, such as by demonstrating that a majority of individuals in public areas of an office building are properly wearing PPE.

Embodiments of the BES 150 can include a BES network interface 310, an administrative interface 315, a protocol director 320, a storage subsystem 325, an entity aggregator 330, a BES sensor network 335, an aggregation locator 340, a protocol exclusion monitor 350, a scoring engine 355, and a compliance engine 360. In some embodiments, the BES 150 can be implemented as a cloud service, or other distributed service. Embodiments of the BES network interface 310 can include any suitable features and components to enable communicative coupling with user mobile devices 105 and/or LADs 130 via the one or more networks 120. In some embodiments, the BES 150 is a dedicated BES 150 for a particular office building or other localized entity. In other embodiments, the BES 150 services multiple entities, and/or a larger geographical area. For example, a single BES 150 can be in communication with LADs 130 in multiple branch locations of an organization across a wide geographical area, or a single BES 150 can service an entire state.

Embodiments of the protocol director 320 include any components or features to facilitate definition of a social distancing protocol and/or other related information. In some embodiments, the protocol director 320 is coupled with the administrative interface 315 to enable an administrator (e.g., any authorized individual or entity) to access the protocol director 320 to confirm, update, modify, delete, or otherwise interface with protocol definitions handled by the protocol director 320. The administrative interface 315 can include, or have access to, any suitable interface infrastructure, such as a web portal, application, user interface, physical or logical ports, etc. The protocol director 320 can include any suitable protocol definition information. In some implementation, a minimal definition is provided, such as a prescribed minimum distance (e.g., six feet) that is generally enforceable. In other implementations, the protocol definition includes information relevant to the manner in which a pathogen spreads, such as information relating to mode of transmission (e.g., whether the pathogen tends to spread through direct contact, through the air, through bodily fluids, through animals, etc.), information relating to environmental factors (e.g., whether the pathogen's spread tends to be affected by changes in, or ranges of, temperature, humidity, airflow, etc.), information relating to host factors (e.g., whether the pathogen's spread tends to correlate with an individual's age, general health, past exposure to the same or a related pathogen, vaccination record, etc.), information relating to pathogen factors (e.g., the pathogen's typical incubation time between infection and appearance of symptoms, basic reproduction number (e.g., an average number of people likely to be infected by any single infected individual, sometimes referred to as “R0”), death rate, etc.), and/or any other relevant information. In some cases, the protocol definition is developed (e.g., defined, recommended, etc.) by, or in conjunction with, an official health organization. In some implementations, the official health organization can directly apply the definition to the protocol director 320 via access to the administrative interface 315. In some embodiments, the protocol definitions are maintained only at the storage subsystem 325. In other embodiments, some or all of the protocol definition can be pushed out to user mobile devices 105 and/or LADs 130, and/or pulled (e.g., requested) by those devices at suitable times.

In some embodiments, the protocol director 320 includes definitions of various exclusions, for example, as exclusion handling rules. Such rules can define cases, in which an otherwise non-compliant action is excluded from non-compliance, considered excusable, not intended to be included as non-compliance, etc. As one example, a social distancing protocol definition may state that individuals must stay at least two meters from each other to remain in compliance. Exclusions may be defined to exclude cases where a solid physical barrier (e.g., a wall or window) is determined to be between the individuals, where the individuals are part of a single household, where the individuals are wearing sufficiently protective PPE, or where the individuals have already spent a sufficient amount of time in close proximity in another setting, such as carpooling or riding on an airplane or public transit together, and the like.

Embodiments of the entity aggregator 330 include any suitable hardware and/or software to aggregate data from multiple user mobile devices 105 and/or multiple entities (e.g., from one or more LADs 130) at a single time, over a time window, and/or in any suitable manner. Various levels of aggregation can be supported by architecting BESs 150 and/or LADs 130 in a hierarchy, or other suitable logical structure. For example, a very large number of user mobile devices 105 are locally aggregated by respective ones of a smaller number of LADs 130, groups of LADs 130 communicate with respective ones of a small number of BESs 150 (or other LADs 130), and the small number of BESs 150 (or other LADs 130) communicates with one or more centralized BES nodes. Depending on the architecture, the entity aggregator 330 may send raw (non-aggregated) data to an upstream device (e.g., another BES 150 at a higher aggregation level), the entity aggregator 330 may aggregate data and send aggregated data to the upstream device, the entity aggregator 330 may store the non-aggregated and/or aggregated data (e.g., in the storage subsystem 325), etc. In some embodiments, the entity aggregator 330 can process the data as part of aggregation, for example, by time-synchronizing or time-normalizing the data, grouping data by location, grouping data by time window, filtering for protocol-related events, tagging the data with metadata, etc. In some embodiments, the entity aggregator 330 (or the local aggregation engine of the LAD 130) can affiliate user mobile devices 105 with individual users. For example, one individual may be affiliated with multiple user mobile device 105 (e.g., a smart phone, a smart watch, etc.), and embodiments can aggregate the devices to the user to help ensure that the individual is only counted once in compliance data, that the individual is able to use different types of devices at different times without being out of compliance, etc.

Aggregated (and/or raw) data, and/or other suitable data, can be stored by the storage subsystem 325. Embodiments of the storage subsystem 325 can include any suitable types of data storage for storing the various types of data, as described herein. For example, the storage subsystem 325 can include remote storage (e.g., a remote server), distributed storage (e.g., cloud-based storage), local storage (e.g., one or more solid-state drives, hard disk drives, tape storage systems, etc.). Though not explicitly shown, user mobile devices 105 and/or LADs 130 can additionally or alternatively be used to store certain types of data relating to features described herein. For example, an individual's user mobile device 105 can locally store personal information about the individual, such as identifying information, health-related information, location tracking information, etc.; and/or LADs 130 can store aggregated sets of such information. Various types of information may be stored, as various types of information may have relevance to an individual's susceptibility to a pathogen, an individual's likelihood of transmitting a pathogen, an individual's likelihood of exhibiting symptoms or of experiencing health complications due to a pathogen, and/or other factors. For example, relevant information can include age, vaccination status, activity level, past infection information, known or suspected associations with other individuals, knowledge level, historic location information, etc. These and other types of data can be stored by the storage subsystem 325 (and/or other storage of user mobile devices 105 and/or LADs 130) in any suitable manner, such as using any suitable type of data structures and/or using any suitable types of data security. In some embodiments, various anonymization techniques are used, for example, to comply with privacy policies and/or regulatory regimes (e.g., the European Union's General Data Protection Regulation 2016/679 (GDPR), the United States' Health Insurance Portability and Accountability Act of 1996 (HIPAA), etc.). Such embodiments can, for example, encrypt stored data, store anonymized data separate from other data usable to de-anonymize the data, etc.

In at least one embodiment, such aggregated anonymized data may be stored in a blockchain for later access. For example, a user mobile device 105 may collect info regarding other devices, and hence users, that it comes in contact with during the day. Such collected info may include a device identifier, such as a media access control (MAC) address, or other identifier for the device or user, along with a location of the encounter, time/day and length of time the devices were in close proximity. That information may be subsequently placed onto a public blockchain for storage and access. Such data may be used for contract tracing in the event that a user is subsequently inflicted with any kind of pathogen. For example, the blockchain may include information that user mobile device 105 was in close proximity with another device with the ID 12345 on May 20, 2020, for approximately 2 minutes while waiting in line nearby at the grocery store. Such data may indicate that the user of UMD 105 came within four feet of the user of device ID 12345. That info can be stored on the blockchain by either of UMD 105 or the other device. If either user is subsequently diagnosed with any type of contagion, then such information can also be placed onto the blockchain to allow either devices to subsequently retrieve such data and compare the info with known location data as part of a contact tracing process.

In some embodiments, the aggregation locator 340 uses the aggregated data to generate aggregation-based location data, and other related information. Some embodiments of the aggregation locator 340 compare location and/or proximity data from multiple user mobile devices 105 to provide additional proximity detection features. As one example, GPS data provided by any particular user mobile device 105 may be accurate only to within a few meters, which may be too imprecise for determining certain social distancing parameters. However, by aggregating proximity data (e.g., using location data from the locator sensors 222, proximity data from the proximity sensors 224, etc.) for multiple user mobile devices 105 that are close to each other, the aggregation locator 340 can achieve better location resolution (e.g., by triangulating, or the like).

As another example, a first user mobile device 105 can determine that it is proximate (e.g., less than six feet from) a second user mobile device 105, but the first user mobile device 105 may have no practical way of determining a precise location of the second user mobile device 105, in which direction is the second user mobile device 105 (e.g., where the first and/or second user mobile device 105 is obscured by a barrier, or the like), an identity of the individual associated with the second user mobile device 105, etc. Such a case can be illustrated by user 102f in FIG. 5. It may be that user 102f is sufficiently distanced from user 102e to comply with a social distancing protocol; and user 102f is too close to user 102j to comply with a social distancing protocol, but is separated by a wall preventing any pathogen communication between users 102f and 102j). If the user mobile device 105 of user 102f buzzes, or otherwise indicates that user 102f is too close to another user, user 102f may assume that he needs to move even farther from user 102e, which is not correct, may not be practical (e.g., the office size may not permit more distance), and may actually move user 102f even closer to an undesirable condition. If the aggregation locator 340 can determine that the concern is between users 102f and 102j and can inform one or both of those users' user mobile devices 105, those users may know better how to respond. For example, in response to user 102f receiving a warning that he is too close to user 102j (or to a user located “behind” him), user 102f may look around, realize that user 102j is not in the room (or that, as his back is to the wall, there is no user “behind” him), and may ignore the warning (or interact with the user mobile device 105 to indicate that there is not really a concern, for example, to help train machine learning algorithms for protocol exclusions, to improve compliance monitoring, etc.). Another such case can be illustrated by the parent and children playing ball together in FIG. 4. The family members may all be within less than a protocol-prescribed distance of each other, which may cause the user mobile device 105 of any or all of the family members to buzz, or otherwise indicate the condition. However, by receiving additional information generated by the aggregation locator 340 indicating that the condition involves family members, they may ignore the indications. Alternatively, the condition may actually involve someone other than another family member (e.g., someone passing on the path, the person under the tree, etc.). With only a generic indication of non-compliance with a social distancing protocol, the family members may assume the non-compliance only involves the other family members, and they may fail to look around to see if another possible non-compliant condition exists.

Some embodiments of the aggregation locator 340 include a location modeler 342, which can further use aggregated location data to generate and use location models to provide additional features. In some embodiments, the location modeler 342 tracks location changes over time to determine direction of movement of one or more individuals, speed of movement of one or more individuals, and/or other similar information. Some such embodiments use direction, speed, and/or other such information to model individual and/or group movement patterns, such as for prediction of impending or future scenarios in which a non-compliant condition is likely to occur. Other such embodiments use location, direction, speed, and/or other such information to model tendencies of individuals and/or groups of individuals to be in certain locations at certain times, to affiliate with other individuals and/or groups, to spend certain amounts of time in certain locations and/or around particular other individuals, etc. Other such embodiments use location, direction, speed, and/or other such information to model and/or estimate location characteristics, such as whether an individual appears to be in a private vehicle, a public vehicle, a private residence, an office, a public place, an indoor location, an outdoor location, a highly trafficked location, a sparsely populated location, etc. All of these and/or other types of information, models, etc. can be used to inform aspects of compliance, application of exclusions, effectiveness of protocol definitions and/or communication of those definitions, etc.

Some embodiments of the aggregation locator 340 include a heat mapper 344. Embodiments of the heat mapper 344 use aggregated location data to determine past, present, and/or future regions of concern, such as regions in which non-compliant situations are common, likely to occur, etc. As one example, prior to walking a certain route along a path or hallway, prior to using a public restroom, prior to visiting or entering a store or public location, and/or in any other suitable scenario, a user can access an application 212 via a user mobile device 105 to see whether that location is presently at a high risk for non-compliance. The result may be generated according to heat map data computed by the heat mapper 344, indicating the high risk for non-compliance as due to the location having a high population density, having a low occurrence of people using PPE, etc. As another example, heat maps generated by the heat mapper 344 can contribute to determining whether there has been individual and/or organizational compliance with a social distancing protocol. For example, such heat maps can indicate whether, when, how often, etc. individuals are congregating in certain areas and in certain ways.

As described herein, there can be many factors that limit the applicability of generalized social distancing protocol definitions. Embodiments of the protocol exclusion monitor 350 use various types of information to automatically apply, generate, and/or modify (e.g., remove, apply, adapt, adjust, tailor, etc.) exclusion rules for a protocol definition controlled by the protocol director 320. For example, an airborne pathogen cannot be communicated from one individual to another if they are completely separated by a solid barrier (e.g., a wall, window, etc.), even if they are in very close proximity to each other. As another example, some pathogens tend only to be dangerous to certain portions of a population (e.g., elderly, infants, people with compromised immune systems, etc.). As another example, some pathogens tend to spread much more easily in certain environments, such as in certain ambient temperatures, certain ambient humidity levels, certain wind conditions, etc. As another example, a set of people has had substantial and repeated contact with the other members of the set, there may be no practical purpose to enforcing separation between the individuals of that set, such as in the carpooling example described above. As another example, once an individual has been vaccinated for a certain pathogen, they may be treated differently in some instances. As another example, certain protocol definitions may not be enforced, or may be relaxed, when individuals are wearing PPE, known to be in a controlled environment, etc. These and many other considerations can inform certain exclusions to the manner in which a social distancing protocol is enforced or applied. In some embodiments, as described above, such exclusions and/or rules relating to such exclusions can be explicitly provided to the protocol director 320 as part of the protocol definition. In other embodiments, some or all such exclusions and/or rules relating to such exclusions can be automatically developed over time based on machine learning, statistical modeling, and/or other techniques. Embodiments of the protocol exclusion monitor 350 can direct other components of the BES 150 based on the exclusions. For example, the protocol exclusion monitor 350 can exclude warnings from being generated for otherwise non-compliant behaviors, can provide nuance to compliance monitoring (e.g., to ensure that an organization is not penalized for non-compliance in cases where an exception should be applied), to adjust scoring (e.g., as described below), etc. In some cases, the exclusion monitor 350 may be configured to identify multiple devices associated with a particular user for exclusion, such as a mobile phone, watch, and tablet, that can all be grouped together and associated with a single user.

In some embodiments, the protocol exclusion monitor 350 includes, or has access to, mapping data (e.g., street maps, architectural maps, three-dimensional building maps, etc.). The mapping data can be used to effectively establish geo-fencing, location boundaries, or location-specific exclusions. As one example, applying the mapping data to a present location of one or more user mobile devices 105 can inform whether a user mobile device 105 is presently indoors or outdoors, in a highly populated or sparsely populated environment, in a well-ventilated or poorly ventilated environment, in a private vehicle or a public vehicle, etc. Any or all of these different locations or location categories can be associated with particular exclusion rules for particular social distancing protocols. For example, a protocol definition can indicate a larger minimum social distancing requirement for indoor areas than for outdoor areas, or for poorly ventilated areas than for well-ventilated areas; or a protocol definition can require constant use of PPE in a highly populated area, but require use of PPE in sparsely populated areas only when approaching within a particular distance of another individual. As another example, applying the mapping data can permit exclusions to be automatically established based on detecting that individuals are separated by physical barriers, or the like. For example, for an individual who would otherwise be warned about excessive proximity to another individual, the protocol exclusion monitor 350 may automatically determine that the individuals at issue are on either side of a wall and may instruct an exclusion to be applied (e.g., thereby not warning the individual, or prompting the individual to confirm the existence of the barrier). As another example, applying the mapping data can permit manually suggested exclusions automatically to be confirmed or authorized. For example, in response to an individual being warned about excessive proximity to another individual who is actually in a separate room (i.e., separated by a wall), the individual uses the GUI 214 of the user mobile device 105 to recommend an exclusion based on physical barrier separation. The protocol exclusion monitor 350 can automatically check whether it seems likely that a physical barrier does exist there (e.g., based on mapping data, based on machine learning, based on prior related requests, etc.), and determines automatically whether to authorize and apply the exclusion.

In some embodiments, the protocol exclusion monitor 350 applies, generates, and/or modifies exclusion rules for a protocol definition based on close contact circles. In some such embodiments, individuals can manually provide information to indicate people in their close contact circles. For example, such embodiments may limit close circle contacts to a maximum number of contacts (e.g., no more than ten individuals, no more than to “families,” etc.), to residence (e.g., only individuals residing together), to work group (e.g., only people occupying a particular area of an office complex), etc. In other such embodiments, some or all close circle contacts are administratively applied and/or confirmed. For example, close contact circles are established automatically or manually by an administrative entity based on demographic data (e.g., publicly recorded residence address, family name, etc.), or other suitable data. As another example, an organization may station a particular individual (e.g., a security guard, nurse, or the like) at a building entrance with PPE to record the temperature of every employee or customer entering the building; and the organization may desire to add that particular individual to the close contact circle of all employees and customers (or otherwise provide an exclusion for that particular individual). Providing the ability for administrative exclusion of such an individual can be desirable to an organization for various reasons, such as because it may be annoying for customers and employees to be warned of non-compliance while having their temperature measured for the sake of compliance, because the organization does not wish to be penalized for non-compliant events every time an individual enters the building, etc.

In other such embodiments, machine learning or other techniques are used by the protocol exclusion monitor 350 to automatically generate close contact circles and/or suggest modifications (additions or deletions) to close contact circles. For example, data from the aggregation locator 340 (e.g., the location modeler 342) can be used to determine that a particular individual tends to spend a lot of time in close proximity to a particular other individual, or the like, which can indicate that the individual should be added, or recommended for adding, to the close contact circle of the particular individual. Similarly, data from the aggregation locator 340 (e.g., the location modeler 342) may indicate that, while a particular individual has a family member listed as residing at the same address and presently included in the particular individual's close contact circle, the family member has not been in proximity of the particular individual for an extended amount of time, and should be deleted, or recommended for deletion, from the close contact circle. In some cases, automatic generation or modification of close contact circles can involve categories of users, rather than individual users. For example, for a particular pathogen that is particularly dangerous to people over the age of seventy, the close contact circle may be automatically tailored to ensure that individuals over seventy years old are excluded from the close contact circles, even if they would otherwise be included. As an illustrative example, a particular individual tends to be in close regular contact with thirty different people, one being over seventy years old. The close contact circle will attempt automatically to add each of those thirty individuals over time, and ultimately, the close contact circle will be generated to include only the 29 individuals not over seventy years old. As such, the user mobile device 105 may be automatically excluded from warning the particular individual when too close to any of those other 29 individuals (as each of those individuals is in the close contact circle associated with that user mobile device 105), but the user mobile device 105 will continue to warn the particular individual when too close to the one individual of particular concern.

Some embodiments of the BES 150 include a scoring engine 355 that uses data from other components to compute scoring of protocol-related events, responses, etc. In some embodiments, the scoring engine 355 uses data from the aggregation locator 340, BES sensor network 335, and/or other components to score individual encounters with social distancing protocol-related events. The scoring can indicate whether a particular individual is properly complying with protocol-prescribed social distancing behaviors, properly complying with protocol-prescribed PPE behaviors, properly complying with protocol-prescribed check-in or other reporting protocols (e.g., checking in each morning with a nurse for a temperature and general health check, periodically submitting a self-assessment form, periodically sending a selfie image of the individual using PPE, etc.), opting in to contact tracing, wearing tracking devices, etc. In some implementations, the scoring can include factors based on general health and/or activity scores (e.g., overall fitness level, participation in stress-reducing activities, vaccination, etc.). In some implementations, the scoring can account for contextual factors. As one example, if individual A is standing still, and individual B approaches and causes a non-compliant condition (they are too close together), individual B may be scored more harshly than individual A for the non-compliant interaction. In some implementations, scoring can be impacted by any exclusions (e.g., full exclusions, relaxing of restrictions, selective applications, etc.) applied by the protocol exclusion monitor 350. For example, an individual's scoring may not reflect any individual non-compliance (or may be less impacted) if an exclusion was applied.

Scoring can be used in various ways. In some embodiments, the scores are used for aggregation to further score groups of individuals. For example, the scores can be aggregated to a family level, a work group or business unit level, a neighborhood level, a company level, a branch office level, a state or country level, or any other suitable level. Those aggregated scores can then indicate macro level behaviors, compliance, etc. In some embodiments, the scoring supports various gamification features. As one example, an application 212 on a user mobile device 105 can indicate a user's (or group's, company's, etc.) scores in a manner that incentivized desired protocol-related behaviors, such as by proving rewards for desired behaviors.

Embodiments of the BES 150 include a compliance engine 360 to monitor and/or report compliance with social distancing protocol definitions controlled by the protocol director 320. In some embodiments, compliance is defined by the protocol director 320 as part of a social distancing protocol definition. In other implementations, the compliance engine 360 maintains a compliance definition separate from the protocol definition of the protocol director 320. In some implementations, an individual or organization (e.g., company, state, etc.) is considered in compliance so long as they have no more than a predefined number of non-compliant events (e.g., without applied exceptions). In other implementations, only certain portions of the protocol definition are applicable for compliance. For example, the protocol definition includes a number of compliance actions (e.g., maintaining a minimum social distance) and a number of recommended actions (e.g., use of PPE), and compliance is determined based only (or primarily) on the compliance actions. In other implementations, individual and/or aggregated scores from the scoring engine 355 are applied to compliance definitions to determine compliance. For example, an individual or entity is considered in compliance so long as a minimum score is maintained or exceeded over a compliance window.

Compliance determinations of the compliance engine 360 can be used in various ways. In some embodiments, compliance determinations are generated for use by regulators and/or other compliance authorities. For example, a health ministry, insurance company, or other entity may perform regular compliance audits using the compliance engine 360, be informed of non-compliance by the compliance engine 360, etc. Such compliance results can then be used, for example, to support certain insurance coverage or premiums, to support imposition or removal of sanction, etc. In other embodiments, compliance determinations are generated for use by entities for self-assessment. For example, a business may desire to perform a compliance self-audit for insurance purposes, to monitor the health and behavior of their employees, to determine effectiveness of social distancing protocols and/or their enforcement, to help determine where to deploy resources relating to communication and enforcement of social distancing protocols, to determine whether employees are following proper hygiene and washing protocols, etc. In the example of businesses, the systems described herein can be utilized to identify customers that do not follow posted protocols, such as social distancing, and utilize such info to prohibit such customers from entering the establishment in the future, or during specified times, such as vulnerable population shopping hours.

In some embodiments, compliance determinations and/or scores can be made available to various stakeholders for other purposes. In some such embodiments, compliance and/or scoring data is searchable by consumers (e.g., the general public) through an application 212 on a user mobile device 105. For example, prior to visiting or interacting with a particular store, a consumer can search through the application 212 to discover whether the store is in compliance with social distancing protocols. As another example, a consumer can use an application 212 to search for entities of a certain category, and/or in a particular geographic region, that are compliant with social distancing protocols and/or have higher than a defined score. In some implementations, such data can be combined with heat map data from the heat mapper 344 and/or other data. For example, an individual may see from scoring or compliance data that a particular supermarket has generally poor compliance behavior, but may also see from the heat map data that the supermarket presently has a very low customer density; and the individual may determine that it is a relatively safe time to visit that store. In some such cases, the application 212 or other interfaces can enable organizations to publicize the scores or compliance status to various ends. For example, a store may desire to advertise that it is highly compliant and be able to show the scores or compliance audit results as objective support for that claim.

In at least one embodiment, the heat map data may be utilized to control entrance or capacity of an establishment. For example, if the heat map data shows that appropriate social distancing is not occurring, or that capacity in a location is particularly high, then appropriate measures may be taken to control further admission to the facility. This may include a physical barrier, such as a gate arm or turnstile, or a visual indicator, such as a screen with a stop sign, or other indicators presented on a user's mobile device 105. Such heat map data may be updated in real-time to control entrance to the facility and the appropriate indicators. Similarly, such data can be used to trigger communication of capacity control messages to a user mobile device 105 of an agent of the location, such as a security guard, employee, business owner, police officer, etc. For example, when capacity is met at a restaurant, a user mobile device 105 of a host, or other front-of-house employee, can indicate such, and the employee can then know with to prevent entry to subsequent customers.

In some embodiments, components of the BES 150, such as the protocol exclusion monitor 350, the aggregation locator 340, the compliance engine 360, the scoring engine 355, etc., use sensor data to inform certain models, computations, etc. Embodiments of the BES sensor network 335 be similar to embodiments of the LAD sensor network 380 and can include any sensors to provide or support such features. For example, the protocol exclusion monitor 350 can generate and/or modify exclusions based on images demonstrating that individuals are using or not using PPE, based on ambient weather conditions, etc. Various compliance features of the compliance engine 360 and/or scoring features of the scoring engine 355 can be similarly impacted by sensor data of the BES sensor network 335. In some implementations, some or all of the sensors are included on-board the BES 150 as part of the BES sensor network 335. In other implementations, some or all of the sensors are off-board the BES 150, and they are considered part of the BES sensor network 335 by virtue of the BES sensor network 335 being in communication with those sensors and being capable of receiving (e.g., and processing, aggregating, etc.) data from those sensors. For example, the BES sensor network 335 can include environmental sensors, cameras, etc.

Embodiments of the BES 150 can support additional features and/or components. For example, some embodiments interface with systems and methods for network tracking of contagion propagation through host populations, such as those described in U.S. patent application Ser. No. 16/839,697, filed Apr. 3, 2020, titled “NETWORK TRACKING OF CONTAGION PROPAGATION THROUGH HOST POPULATIONS,” the disclosure of which being hereby incorporated herein in its entirety.

While some embodiments are described above with respect to social distancing protocols relating to the spread of contagious pathogens, embodiments can be used with other types of social distancing protocols. Some embodiments operate in context of restraining orders, and the like. For example, a court can order a particular individual to maintain at least a prescribed minimum distance from another individual or group or category of individuals. Features described herein can be used to define parameters of a corresponding social distancing protocol. Other embodiments operate in context of organizational security, and the like. As one example, a corporation may maintain certain information as proprietary from particular individuals in the organization, individuals outside the organization, customers, competitors, etc. Location and proximity tracking, close contact circles, compliance, and other features described herein can be used to help ensure that unauthorized individuals are not in proximity to areas where they should not be, are not in close proximity to authorized individuals for extended periods of time, etc. Further, if such suspicious or unauthorized contacts do occur, data can be used to facilitate contact tracing and/or other features to determine who else may be suspect, what information or individuals may be compromised, for how long such suspect event have been occurring, etc. Other embodiments operate in other contexts in which portions of a population are intentionally separated from other portions. As one example, features described herein can help inform, track, and enforce certain behaviors relating to a group of quarantined or sequestered individuals and/or others potentially in contact with those quarantined or sequestered individuals. As another example, features described herein can help inform, track, and enforce separation of work groups for various reasons, such as to maintain separate and distinct work environments to support pathogenic social distancing, or the like.

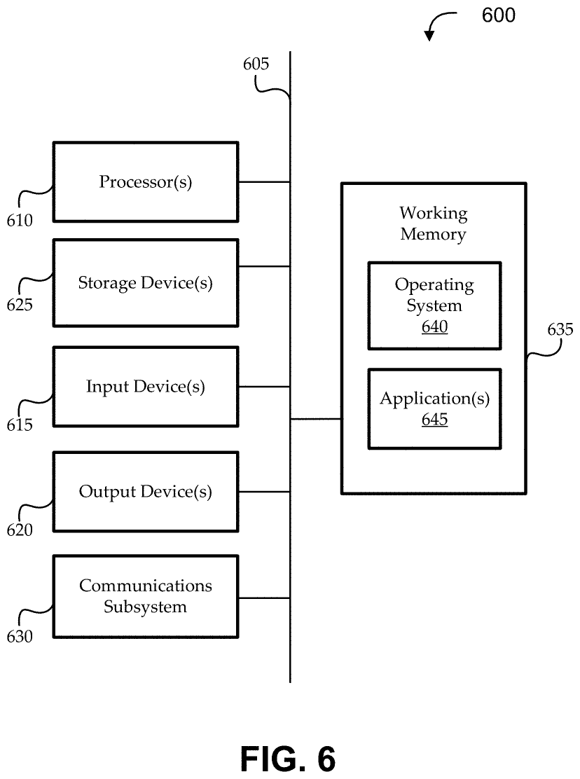

Embodiments of the user mobile device 105, the LAD 130, the BES 150, or components thereof, can be implemented on, and/or can incorporate, one or more computer systems, as illustrated in FIG. 6. FIG. 6 provides a schematic illustration of one embodiment of a computer system 600 that can implement various system components and/or perform various steps of methods provided by various embodiments. It should be noted that FIG. 6 is meant only to provide a generalized illustration of various components, any or all of which may be utilized as appropriate. FIG. 6, therefore, broadly illustrates how individual system elements may be implemented in a relatively separated or relatively more integrated manner.

The computer system 600 is shown including hardware elements that can be electrically coupled via a bus 605 (or may otherwise be in communication, as appropriate). The hardware elements may include one or more processors 610, including, without limitation, one or more general-purpose processors and/or one or more special-purpose processors (such as digital signal processing chips, graphics acceleration processors, video decoders, and/or the like); one or more input devices 615, which can include, without limitation, a mouse, a keyboard, remote control, and/or the like; and one or more output devices 620, which can include, without limitation, a display device, a printer, and/or the like. In some implementations, the computer system 600 is a server computer configured to interface with additional computers (not with human users), such that the input devices 615 and/or output devices 620 include various physical and/or logical interfaces (e.g., ports, etc.) to facilitate computer-to-computer interaction and control.

The computer system 600 may further include (and/or be in communication with) one or more non-transitory storage devices 625, which can comprise, without limitation, local and/or network accessible storage, and/or can include, without limitation, a disk drive, a drive array, an optical storage device, a solid-state storage device, such as a random access memory (“RAM”), and/or a read-only memory (“ROM”), which can be programmable, flash-updateable and/or the like. Such storage devices may be configured to implement any appropriate data stores, including, without limitation, various file systems, database structures, and/or the like.

The computer system 600 can also include a communications subsystem 630, which can include, without limitation, a modem, a network card (wireless or wired), an infrared communication device, a wireless communication device, and/or a chipset (such as a Bluetooth™ device, an 602.11 device, a WiFi device, a WiMax device, cellular communication device, etc.), and/or the like. The communications subsystem 630 can support multiple communication technologies and/or can provide communications with one or more communication networks 160.

In many embodiments, the computer system 600 will further include a working memory 635, which can include a RAM or ROM device, as described herein. The computer system 600 also can include software elements, shown as currently being located within the working memory 635, including an operating system 640, device drivers, executable libraries, and/or other code, such as one or more application programs 645, which may include computer programs provided by various embodiments, and/or may be designed to implement methods, and/or configure systems, provided by other embodiments, as described herein. Merely by way of example, one or more procedures described with respect to the method(s) discussed herein can be implemented as code and/or instructions executable by a computer (and/or a processor within a computer); in an aspect, then, such code and/or instructions can be used to configure and/or adapt a general purpose computer (or other device) to perform one or more operations in accordance with the described methods. In some embodiments, the operating system 640 and the working memory 635 are used in conjunction with the one or more processors 610 to implement some or all of the systems described herein.

For example, in context of the user mobile device 105, the one or more processors 610 can implement the UMD processor 210, and the operating system 640 and the working memory 635 can be used in conjunction with the one or more processors 610 to implement some or all of the applications 212, the GUIs 214, and software components of the UMD sensor network 220, the UMD network interface 230, and the indicator subsystem 240. In context of the LADs 130, the operating system 640 and the working memory 635 can be used in conjunction with the one or more processors 610 to implement some or all of the LAD network interface 370, the local aggregation engine 375, and the LAD sensor network. In context of the BESs 150, the storage devices of the computational system 600 can implement the storage subsystem 325, and the operating system 640 and the working memory 635 can be used in conjunction with the one or more processors 610 to implement some or all of the BES network interface 310, the administrative interface 315, the protocol director 320, the entity aggregator 330, the aggregation locator 340, the protocol exclusion monitor 350, the scoring engine 355, and the compliance engine 360

A set of these instructions and/or codes can be stored on a non-transitory computer-readable storage medium, such as the non-transitory storage device(s) 625 described above. In some cases, the storage medium can be incorporated within a computer system, such as computer system 600. In other embodiments, the storage medium can be separate from a computer system (e.g., a removable medium, such as a compact disc), and/or provided in an installation package, such that the storage medium can be used to program, configure, and/or adapt a general purpose computer with the instructions/code stored thereon. These instructions can take the form of executable code, which is executable by the computer system 600 and/or can take the form of source and/or installable code, which, upon compilation and/or installation on the computer system 600 (e.g., using any of a variety of generally available compilers, installation programs, compression/decompression utilities, etc.), then takes the form of executable code.

It will be apparent to those skilled in the art that substantial variations may be made in accordance with specific requirements. For example, customized hardware can also be used, and/or particular elements can be implemented in hardware, software (including portable software, such as applets, etc.), or both. Further, connection to other computing devices, such as network input/output devices, may be employed.

As mentioned above, in one aspect, some embodiments may employ a computer system (such as the computer system 600) to perform methods in accordance with various embodiments of the invention. According to a set of embodiments, some or all of the procedures of such methods are performed by the computer system 600 in response to processor 610 executing one or more sequences of one or more instructions (which can be incorporated into the operating system 640 and/or other code, such as an application program 645) contained in the working memory 635. Such instructions may be read into the working memory 635 from another computer-readable medium, such as one or more of the non-transitory storage device(s) 625. Merely by way of example, execution of the sequences of instructions contained in the working memory 635 can cause the processor(s) 610 to perform one or more procedures of the methods described herein.

The terms “machine-readable medium,” “computer-readable storage medium” and “computer-readable medium,” as used herein, refer to any medium that participates in providing data that causes a machine to operate in a specific fashion. These mediums may be non-transitory. In an embodiment implemented using the computer system 600, various computer-readable media can be involved in providing instructions/code to processor(s) 610 for execution and/or can be used to store and/or carry such instructions/code. In many implementations, a computer-readable medium is a physical and/or tangible storage medium. Such a medium may take the form of a non-volatile media or volatile media. Non-volatile media include, for example, optical and/or magnetic disks, such as the non-transitory storage device(s) 625. Volatile media include, without limitation, dynamic memory, such as the working memory 635. Common forms of physical and/or tangible computer-readable media include, for example, a floppy disk, a flexible disk, hard disk, magnetic tape, or any other magnetic medium, a CD-ROM, any other optical medium, any other physical medium with patterns of marks, a RAM, a PROM, EPROM, a FLASH-EPROM, any other memory chip or cartridge, or any other medium from which a computer can read instructions and/or code. Various forms of computer-readable media may be involved in carrying one or more sequences of one or more instructions to the processor(s) 610 for execution. Merely by way of example, the instructions may initially be carried on a magnetic disk and/or optical disc of a remote computer. A remote computer can load the instructions into its dynamic memory and send the instructions as signals over a transmission medium to be received and/or executed by the computer system 600.

The communications subsystem 630 (and/or components thereof) generally will receive signals, and the bus 605 then can carry the signals (and/or the data, instructions, etc., carried by the signals) to the working memory 635, from which the processor(s) 610 retrieves and executes the instructions. The instructions received by the working memory 635 may optionally be stored on a non-transitory storage device 625 either before or after execution by the processor(s) 610.

It should further be understood that the components of computer system 600 can be distributed across a network. For example, some processing may be performed in one location using a first processor while other processing may be performed by another processor remote from the first processor. Other components of computer system 600 may be similarly distributed. As such, computer system 600 may be interpreted as a distributed computing system that performs processing in multiple locations. In some instances, computer system 600 may be interpreted as a single computing device, such as a distinct laptop, desktop computer, or the like, depending on the context.

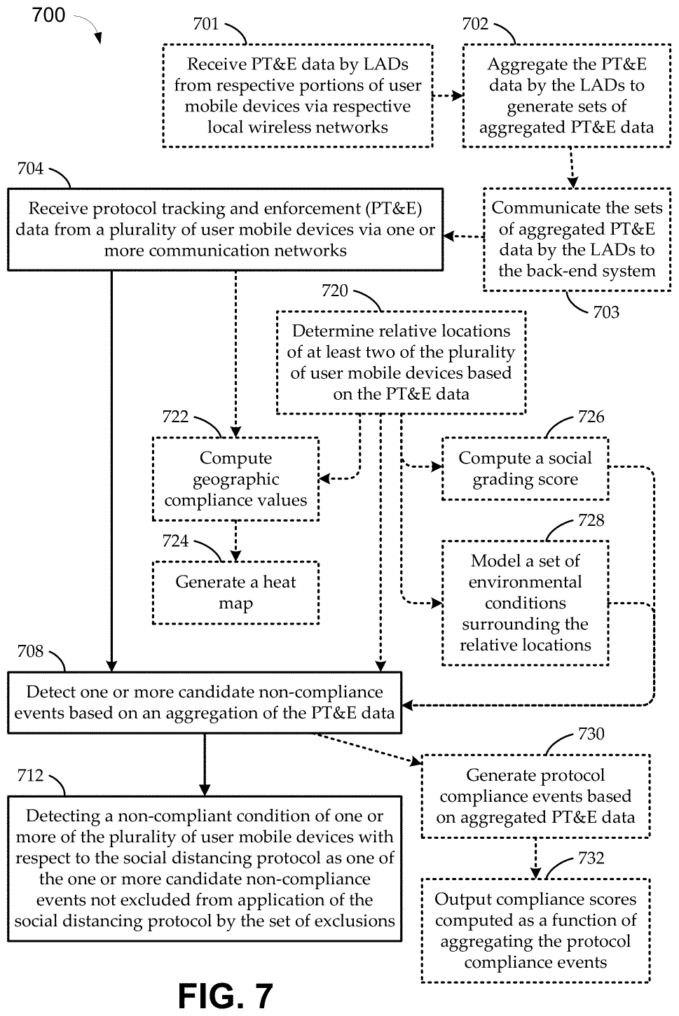

FIG. 7 shows a flow diagram of an illustrative method 700 for tracking of social distancing protocols to mitigate proximity-based communicability, according to various embodiments. As described herein, some embodiments of the method 700 are configured for social distancing protocols the mitigate proximity-based communicability of pathogens, including infectious diseases, and the like. Other embodiments of the method 700 are configured for social distancing protocols the mitigate proximity-based communicability of sensitive information and/or any other undesired or carefully controlled communication. Embodiments of the method 700 can be implemented using any of the systems described here and/or any other suitable systems.

Embodiments can begin at stage 704 by receiving (e.g., at a back-end system) protocol tracking and enforcement (PT&E) data from a plurality of user mobile devices via one or more communication networks. The PT&E data is associated with at least one pre-defined social distancing protocol stored in association with a set of exclusions to application of the social distancing protocol, as described herein. In some embodiments, the receiving is directly from some or all of the user mobile devices. For example, some or all of the user mobile devices are in direct communication with the back-end system.

Some embodiments can begin at stage 701 by receiving, by each of multiple local aggregator devices (LADs), PT&E data from a respective portion of the plurality of user mobile devices via a respective local wireless network. For example, each LAD provides its own WLAN, or other network. At stage 702, such embodiments can aggregate, by each of the plurality of LADs, the PT&E data from the respective portion of the plurality of user mobile devices to generate a respective set of aggregated PT&E data. The aggregation can be for all user mobile devices in communication with that particular LAD, for a particular time window, for a particular subset of user mobile devices (e.g., those belonging to a particular work group, department, business, etc.), etc. At stage 703, such embodiments can communicate the respective sets of aggregated PT&E data by the LADs to the back-end system.

Embodiments can continue at stage 708 by detecting (e.g., by the back-end system) one or more candidate non-compliance events based on an aggregation of the PT&E data. As described herein, detection of compliance or non-compliance depends on the definition of the social distancing protocol. For example, if a protocol defines that individuals are to maintain at least a minimum distance from each other, non-compliance can be detected responsive to that minimum distance being violated. As another example, if a protocol defines that individuals are to wear a certain type of personal protective equipment (PPE) in certain types of locations and/or conditions, non-compliance can be detected responsive to determining that PPE was not worn in one of those locations and/or conditions. As another example, if a protocol defines a maximum number of individuals permitted to gather in a particular type of location, non-compliance can be detected responsive to determining that more than that number of individuals PPE has gathered in the particular type of location.

In some embodiments, at stage 720, the method 700 can determine relative locations of at least two of the plurality of user mobile devices based on the PT&E data. In some such embodiments, detecting the one or more candidate non-compliance events at stage 708 is based at least on the relative locations of at least two of the plurality of user mobile devices. In some such embodiments, the set of exclusions comprises a defined set of trusted devices in relation to any one or more of the user mobile devices, the trusted devices being excluded from consideration for some or all protocol compliance concerns. As one implementation, for a first user mobile device of the plurality of user mobile devices, a respective set of trusted devices defining at least a second user mobile device of the plurality of user mobile devices as permitted to violate the social distancing protocol with respect to the first user mobile device without invoking a non-compliant condition. For example, members of a same household may be permitted to violate a minimum distance requirement, or members of a particular work group may be permitted to gather only with other individuals of that work group.

In some embodiments, at stage 726, a social grading score can be computed for each of the at least two user mobile devices of stage 720. In other embodiments, a social grading score can be computed for any set of user mobile devices. As described herein, the social grading score can be based on an apportionment of non-compliant behavior among the user mobile devices. For example, suppose a first individual approaches a second individual, thereby putting the second individual at risk. Both individuals may trigger detection of a candidate non-compliance event at stage 708, and possibly even a non-compliant condition at stage 712; but the social grading score applied to the first individual can indicate a relatively large apportionment of non-compliant behavior, while the social grading score applied to the second individual can indicate a relatively small apportionment of non-compliant behavior.

Some embodiments, at stage 728, model a set of environmental conditions surrounding the relative locations of multiple user mobile devices based on the PT&E data, such as the at least two user mobile devices of stage 720. At least one of the set of environmental conditions is determinative of at least one of the set of exclusions, such that the detecting at stage 712 that the one of the one or more candidate non-compliance events is not excluded by the set of exclusions is based on the at least one of the set of environmental conditions. For example, as described herein, the modeled environmental conditions can indicate relative speeds and/or directions of movement of individuals, whether individuals are indoors or outdoors, whether an individual is in a public or private vehicle, whether multiple individuals are separated by a physical barrier, etc.

Some embodiments, at stage 722, can compute a compliance value for each of multiple geographical locations based on aggregated location data from the PT&E data. Various techniques for computing compliance values are described herein. The geographical locations can be at any suitable geographical resolution, such as by county, by street, by building, by hallway, etc. At stage 724, some such embodiments can generate a heat map to graphically indicate a past, present, and/or future likelihood of compliance across at least a portion of the plurality of geographical locations. In some implementations, the heat map can include a physical location map, such as a map of a state or a building, with different sub-locations labeled, marked, colored, and/or otherwise graphically modified to indicate compliance value. In other implementations, the heat map can include a spreadsheet, chart, or other graphical layout.

At stage 712, embodiments can detect (e.g., by the back-end system) a non-compliant condition of one or more of the plurality of user mobile devices with respect to the social distancing protocol as one of the one or more candidate non-compliance events not excluded from application of the social distancing protocol by the set of exclusions. As one illustrative case, one of the candidate non-compliance events indicates two user mobile devices being separated by less than a minimum distance defined by the protocol. However, applying the set of exclusions results in this event being excluded from treatment as non-compliance in this instance, and the candidate non-compliance event is determined not to be a non-compliant condition, accordingly. For example, the set of exclusions can indicate that the two user mobile devices in this instance are trusted devices (e.g., part of a same household), located in a category of place where the minimum distance is not applicable (e.g., where the protocol defines a different distances or no minimum distance for indoor versus outdoor spaces, public versus private spaces, or the like).

Some embodiments generate compliance scores, as described herein. For example, at stage 730, embodiments can generate protocol compliance events based on aggregated PT&E data. In some implementations, the protocol compliance events include the candidate non-compliance events of stage 708. At stage 732, some such embodiments can output compliance scores computed as a function of aggregating the plurality of protocol compliance events. For example, the aggregation can be at multiple hierarchical aggregation levels, such that the scoring is for multiple hierarchical aggregation levels. For example, compliance scores can be computed at an individual user level, a logical grouping of individuals level (e.g., a household, family, work group, department, etc.), an entity level (e.g., a company, company branch location, etc.), a micro-geographic level (e.g., a street, neighborhood, building, etc.), a macro-geographic level (e.g., county, state, country, etc.), and/or at any other suitable aggregation level. In some implementations, the aggregation level is defined by the social distancing protocol. For example, the protocol can include its own compliance metrics, and the aggregation can be in accordance with those metrics.

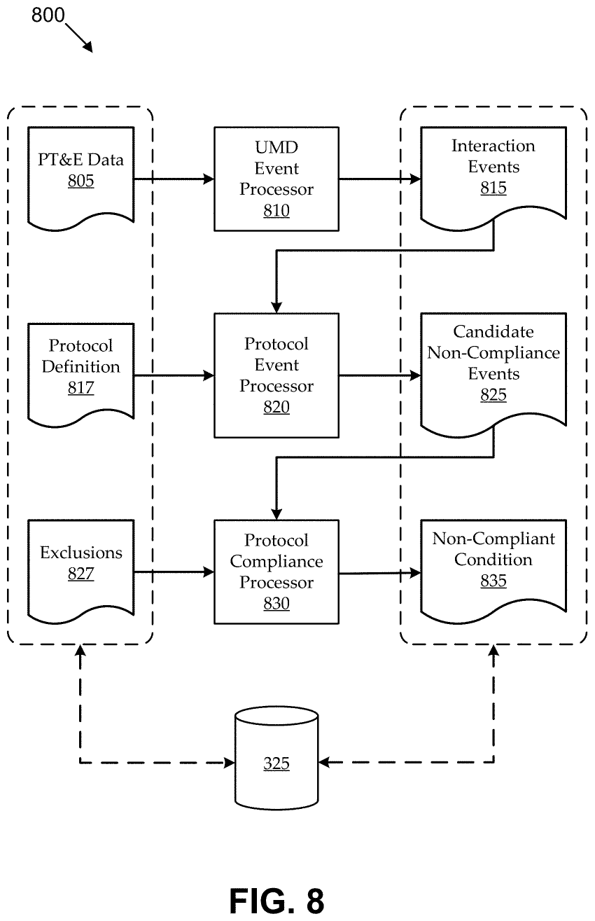

In the various embodiments described above, protocol compliance events can be computed by a series of computational processes. FIG. 8 shows an illustrative simplified computational system 800 for a computing protocol compliance events and related data, according to various embodiments. As illustrated, the computational system 800 includes a user mobile device (UMD) event processor 810, a protocol event processor 820, and a protocol compliance processor 830. In some embodiments, the computational system 800 is implemented by the computational system 600 of FIG. 6. For example, the UMD event processor 810, protocol event processor 820, and protocol compliance processor 830 are processes implemented in working memory 635 and performed by processor(s) 610. In some embodiments, the computational system 800 is an implementation of, or is implemented by, compliance engine 360 of FIG. 3.

Embodiments of the UMD event processor 810 compute interaction events 815 from PT&E data 805. In some implementations, the PT&E data 805 is received directly from the user mobile devices 105. In other implementations, the PT&E data 805 is aggregated (e.g., by the user mobile devices 105, by one or more LADs 130, by the entity aggregator 330 of the back-end system 150, etc.) prior to being received by the UMD event processor 810. In some embodiments, the UMD event processor 810 computes the interaction events 815 without any consideration of the social distancing protocol, or any related definitions. For example, the UMD event processor 810 monitors and analyzes large amounts of data from the user mobile devices 105 to determine any of a broad class of interactions between user mobile devices 105, such as detecting that a particular user mobile device 105 is approaching, or has approached, another user mobile device 105; that a particular user mobile device 105 has entered, or is approaching, a geographic area where gatherings tend to occur; etc. The interaction events 815 can include any present and/or predicted impending conditions (e.g., interactions of any user mobile device with any one or more other user mobile device) that potentially have relevance to protocol tracking, compliance, enforcement, scoring, etc. As discussed herein, some implementations can involve very large numbers of user mobile devices 105, such that there are potentially very large numbers of interaction events 815 taking place at any moment. Some implementations of the UMD event processor 810 implement various bag data computations, machine learning algorithms, artificial neural networks, and/or other techniques to cull the large amounts of user mobile device 105 information for detection of patterns indicating potentially relevant interaction events 815.

Not involving the specifics of a particular protocol definition at this stage can provide various features. One feature is that the UMD event processor 810 can be optimized for consistent and efficient processing of the large amounts of data without re-tooling processing for different protocols, without requiring access to data stores having protocol definitions, without requiring updates and/or external access to the processors implementing the UMD event processor 810, etc. Another feature is that the interaction events 815 can be stored for later analysis, even if they are not ultimately considered relevant to a currently in-force protocol. For example, as scientists, researchers, and/or policy makers determine which protocols are effective, how to change protocols, etc.; large databases of past interaction events 815 can be used to run simulations, as training data for machine learning algorithms, etc. Another feature is that the same UMD event processor 810 can be used concurrently in conjunction with multiple protocols. The same UMD event processor 810 can be coupled with multiple instances of the protocol event processor 820 and/or protocol compliance processor 830, so that the same set of interaction events 815 can be analyzed against different social distancing protocols at the same time. For example, a business may want to use the same interaction events 815 to detect year-round compliance with protocols relating to spreads of common pathogens (e.g., monitoring handwashing, and/or other best practices for limiting spread of colds, flus, etc.), year-round compliance with protocols relating to spread of sensitive information in the company, and also temporary (e.g., on-demand) compliance with protocols relating to spreads of particular pathogens (e.g., novel, highly contagious pathogens, etc.).

Embodiments of the protocol event processor 820 can compute candidate non-compliance events 825 based on the interaction events 815 and the protocol definition 817 of the social distancing protocol. For example, the protocol event processor 820 is coupled with the UMD event processor 810 to receive the computed interaction events 815, and is coupled with the data store 325 of FIG. 3 to receive the protocol definition 817. As described herein, the protocol definition 817 can include any suitable protocols for mitigating proximity-based communicability (e.g., of a pathogen), such as minimum distancing between individuals, limitations on gathering sizes, requirements for use of PPE, etc. The protocol event processor 820 analyzes the interaction events 815 against these protocols to determine which interaction events 815 is a candidate non-compliance event 825. For example, the interaction events 815 may indicate that a particular user mobile device 105 is in proximity to (e.g., in a same building as, in a same conference room as, etc.) twelve other user mobile devices 105; the protocol event processor 820 determines that only one of those interaction events 815 is a candidate non-compliance event 825, as only one of those proximity events is one in which the other user mobile devices 105 is less than six feet away, as defined by the protocol definition 817. As another example, the interaction events 815 may indicate fifty user mobile devices 105 as being “out in public” (e.g., away from their respective home locations) where PPE could potentially be a requirement of a protocol (but may or may not be a requirement of the particular protocol that is currently in effect); the protocol event processor 820 determines that only ten of those interaction events 815 are candidate non-compliance events 825, as only ten of those user mobile devices 105 are in public areas where PPE is, in fact, required, as defined by the protocol definition 817.

Embodiments of the protocol compliance processor 830 can compute non-compliant conditions 835 based on the candidate non-compliance events 825 and set of exclusions 827 defined for the social distancing protocol. For example, the protocol compliance processor 830 is coupled with the protocol event processor 820 to receive the computed candidate non-compliance events 825, and is coupled with the data store 325 of FIG. 3 to receive the set of exclusions 827. The protocol compliance processor 830 then determines whether a particular candidate non-compliance event 825, or grouping of candidate non-compliance events 825, should be considered as a non-compliant condition 835; or whether it should be excluded from consideration based on application of one or more exclusions 827. As described herein, the set of exclusions 827 can include any suitable exclusions to application of the protocol definition 817. Some exclusions 827 are device-related, such as some or all particular user mobile devices 105 being associated with respective trusted devices for which some or all aspects of the protocol definition 817 are not applied. Other exclusions 827 are location-related, such as defining that some or all aspects of the protocol definition 817 are not applied in certain categories of location (e.g., in an individual's home, in outdoor areas, in an office building, in an elevator, etc.). Some exclusions 827 are complete, such that application of the exclusion results in the protocol definition 817 not being applied at all. For example, when a user mobile device 105 is in proximity to another user mobile device 105 associated with an individual living in their same household, an exclusion 827 may indicate that any related candidate non-compliance event 825 (e.g., violation of a protocol-defined minimum separation distance, failure to wear proper PPE, etc.) is excluded from consideration as a non-compliant condition 835. Other exclusions 827 define partial or modified application of the protocol definition 817. For example, the protocol definition 817 may include different protocols for different types of locations (e.g., a first defined maximum number of individuals gathering indoors, and a second defined maximum number of individuals gathering outdoors), such that the exclusion 827 may differently apply the protocol definition 817, and the protocol compliance processor 830 may differently determine whether to consider a candidate non-compliance event 825 as a non-compliant condition 835 based on the type of location (e.g., a determination of the when a location modeler 342 of FIG. 3).

As illustrated, some or all of the data used by and/or generated by FIG. 8 can be stored by the storage subsystem 325. In some embodiments, the storage system 325 stores the protocol definition 817 and the set of exclusions 827. In other embodiments, the storage system 325 further stores some or all of the PT&E data 805. In other embodiments, the storage system 325 further stores some or all of the interaction events 815, the candidate non-compliance events 825, and/or the non-compliant conditions 835. As described herein, these different types of data can be used to support various features. For example, the candidate non-compliance events 825 can be generally stored as protocol events for computing compliance scoring, social grading, and/or other features.

The methods, systems, and devices discussed above are examples. Various configurations may omit, substitute, or add various procedures or components as appropriate. For instance, in alternative configurations, the methods may be performed in an order different from that described, and/or various stages may be added, omitted, and/or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Also, configurations may be described as a process which is depicted as a flow diagram or block diagram. Although each may describe the operations as a sequential process, many of the operations can be performed in parallel or concurrently. In addition, the order of the operations may be rearranged. A process may have additional steps not included in the figure. Furthermore, examples of the methods may be implemented by hardware, software, firmware, middleware, microcode, hardware description languages, or any combination thereof. When implemented in software, firmware, middleware, or microcode, the program code or code segments to perform the necessary tasks may be stored in a non-transitory computer-readable medium such as a storage medium. Processors may perform the described tasks.

Having described several example configurations, various modifications, alternative constructions, and equivalents may be used without departing from the spirit of the disclosure. For example, the above elements may be components of a larger system, wherein other rules may take precedence over or otherwise modify the application of the invention. Also, a number of steps may be undertaken before, during, or after the above elements are considered.

What is claimed is:

1. A system for network tracking of social distancing protocols to mitigate proximity-based communicability, the system comprising:
a back-end system comprising:
a protocol director to define a social distancing protocol;
an entity aggregator configured to receive protocol tracking and enforcement (PT&E) data from a plurality of user mobile devices;
a protocol exclusion monitor to generate a set of exclusions to application of the social distancing protocol; and
a compliance engine to detect a non-compliant condition of one or more of the plurality of user mobile devices with respect to the social distancing protocol based on detecting one or more candidate non-compliance events based on an aggregation of the PT&E data and detecting the non-compliant condition as one of the one or more candidate non-compliance events not excluded from application of the social distancing protocol by the set of exclusions.

2. The system of claim 1, further comprising:
an aggregation locator to determine relative locations of at least two of the plurality of user mobile devices based on the PT&E data,
wherein the compliance engine is to detect the one or more candidate non-compliance events based at least on the relative locations of at least two of the plurality of user mobile devices.

3. The system of claim 2, wherein the set of exclusions comprises, for a first user mobile device of the plurality of user mobile devices, a respective set of trusted devices defining at least a second user mobile device of the plurality of user mobile devices as permitted to violate the social distancing protocol with respect to the first user mobile device without invoking a non-compliant condition.

4. The system of claim 2, further comprising:
a scoring engine to compute a social grading score for each of the at least two of the plurality of user mobile devices in association with detecting the one or more candidate non-compliance events, the social grading score based on an apportionment of non-compliant behavior among the at least two of the plurality of user mobile devices.

5. The system of claim 2, wherein the aggregation locator comprises:
a location modeler configured to model a set of environmental conditions surrounding the relative locations of the at least two of the plurality of user mobile devices based on the PT&E data, at least one of the set of environmental conditions being determinative of at least one of the set of exclusions,
wherein the compliance engine is to detect that the one of the one or more candidate non-compliance events is not excluded by the set of exclusions based on the at least one of the set of environmental conditions.

6. The system of claim 5, wherein the set of environmental conditions indicates one or more of relative directions of movement of the at least two of the plurality of user mobile devices, relative speeds of movement of the at least two of the plurality of user mobile devices, whether one or more of the at least two of the plurality of user mobile devices is separated from others of the at least two of the plurality of user mobile devices by a physical barrier, a population density surrounding the at least two of the plurality of user mobile devices, whether one or more of the at least two of the plurality of user mobile devices is in a private domain, or whether one or more of the at least two of the plurality of user mobile devices is in a public domain.

7. The system of claim 1, further comprising:
a heat mapper configured to compute a compliance value for each of a plurality of geographical locations based on aggregated location data from the PT&E data, and to generate a heat map to graphically indicate a past, present, and/or future likelihood of compliance across at least a portion of the plurality of geographical locations.

8. The system of claim 1, further comprising:
a storage subsystem configured to store the PT&E data in a blockchain, such that the blockchain indicates an aggregated chain of PT&E events, each PT&E event associated with a respective event timing, a respective event location, and at least one respective anonymized event participant.

9. The system of claim 1, further comprising:
a storage subsystem comprising at least one non-transient storage device having the social distancing protocol and the set of exclusions stored thereon.

10. The system of claim 1, further comprising:
one or more local aggregator devices (LADs), each comprising:
a LAD network interface configured to provide a respective local wireless network and to communicatively couple with a respective portion of the plurality of user mobile devices via the respective local wireless network; and
a local aggregation engine configured to aggregate data for the respective portion of the plurality of user mobile devices,
wherein the LAD network interface is further configured to communicate the aggregated data for the respective portion of the plurality of user mobile devices with the back-end system.

11. The system of claim 10, wherein:
the back-end system is remote from the one or more LADs and from the plurality of user mobile devices; and
each of the one or more LADs is configured to communicate the aggregated data with the back-end system via at least one communication network other than the respective local wireless network.

12. The system of claim 1, wherein the compliance engine is further configured to generate a plurality of protocol compliance events based on aggregated PT&E data, and the system further comprises:
a scoring engine to output a plurality of compliance scores computed as a function of aggregating the plurality of protocol compliance events at each of a plurality of hierarchical aggregation levels.

13. A method for network tracking of social distancing protocols to mitigate proximity-based communicability, the system comprising:
receiving, at a back-end system, protocol tracking and enforcement (PT&E) data from a plurality of user mobile devices via one or more communication networks,
the PT&E data associated with at least one pre-defined social distancing protocol stored in association with a set of exclusions to application of the social distancing protocol;
detecting, by the back-end system, one or more candidate non-compliance events based on an aggregation of the PT&E data; and
detecting, by the back-end system, a non-compliant condition of one or more of the plurality of user mobile devices with respect to the social distancing protocol as one of the one or more candidate non-compliance events not excluded from application of the social distancing protocol by the set of exclusions.

14. The method of claim 13, further comprising:
determining relative locations of at least two of the plurality of user mobile devices based on the PT&E data,
wherein the detecting the one or more candidate non-compliance events is based at least on the relative locations of at least two of the plurality of user mobile devices.

15. The method of claim 14, wherein the set of exclusions comprises, for a first user mobile device of the plurality of user mobile devices, a respective set of trusted devices defining at least a second user mobile device of the plurality of user mobile devices as permitted to violate the social distancing protocol with respect to the first user mobile device without invoking a non-compliant condition.

16. The method of claim 14, further comprising:
computing a social grading score for each of the at least two of the plurality of user mobile devices in association with detecting the one or more candidate non-compliance events, the social grading score based on an apportionment of non-compliant behavior among the at least two of the plurality of user mobile devices.

17. The method of claim 14, further comprising:
modeling a set of environmental conditions surrounding the relative locations of the at least two of the plurality of user mobile devices based on the PT&E data, at least one of the set of environmental conditions being determinative of at least one of the set of exclusions,
wherein the detecting that the one of the one or more candidate non-compliance events is not excluded by the set of exclusions is based on the at least one of the set of environmental conditions.

18. The method of claim 13, further comprising:
computing a compliance value for each of a plurality of geographical locations based on aggregated location data from the PT&E data; and
generating a heat map to graphically indicate a past, present, and/or future likelihood of compliance across at least a portion of the plurality of geographical locations.

19. The method of claim 13, further comprising:
receiving, by each of a plurality of local aggregator devices (LADs), PT&E data from a respective portion of the plurality of user mobile devices via a respective local wireless network;
aggregating, by each of the plurality of LADs, the PT&E data from the respective portion of the plurality of user mobile devices to generate a respective set of aggregated PT&E data; and
communicating the respective sets of aggregated PT&E data by the plurality of LADs to the back-end system.

20. The method of claim 13, further comprising:
generating a plurality of protocol compliance events based on aggregated PT&E data, the plurality of protocol compliance events comprising the one or more candidate non-compliance events; and
outputting a plurality of compliance scores computed as a function of aggregating the plurality of protocol compliance events at each of a plurality of hierarchical aggregation levels.
